(19) United States
(12) Patent Application Publication
(10) Pub. No.: US 2022/0080106 A1
(43) Pub. Date: Mar. 17, 2022

(54) INTRAVASCULAR OXYGENATION SYSTEM AND METHOD

(71) Applicant: Agitated Solutions Inc., Oakdale, MN (US)

(72) Inventors: Morgan Evans, Apple Valley, MN (US); Benjamin Arcand, Minneapolis, MN (US); Carl Lance Boling, San Jose, CA (US); Jennifer Chmura, Minneapolis, MN (US)

(73) Assignee: Agitated Solutions Inc., Oakdale, MN (US)

(21) Appl. No.: 17/464,258

(22) Filed: Sep. 1, 2021

Related U.S. Application Data
(60) Provisional application No. 63/073,063, filed on Sep. 1, 2020.

(51) Int. Cl. A61M 5/00; A61M 37/00; A61M 39/24; A61M 25/00

(52) U.S. Cl. CPC A61M 5/00; A61M 37/0092; A61M 2202/0208; A61M 25/0068; A61M 2005/006; A61M 39/24

(57) ABSTRACT

A system for intravascular oxygenation may include a catheter shaft, a vibratory member, and an oxygen source. The catheter shaft may have a wall that extends from a proximal end to a distal end along a longitudinal axis to form a lumen. The distal end may terminate in an atraumatic tip that seals off an interior space of the lumen from an adjacent exterior space. The distal end may include a coiled spring whose coils are tightly disposed against adjacent coils. The vibratory member may be configured to produce and transmit via the wall, to the coiled spring, mechanical vibration or high-frequency acoustic energy. The oxygen source may be configured to be coupled to the proximal end and to deliver a flow of oxygen to an interior space for communication to the exterior space, through gaps that exist or are created between adjacent coils of the coiled spring.

This application claims the benefit of U.S. Provisional Application Ser. No. 63/073,063, titled “Intravascular Oxygenation System and Method,” filed on Sep. 1, 2020. This application incorporates the entire contents of the foregoing application herein by reference.

### TECHNICAL FIELD

Various embodiments relate generally to systems and methods for supplementing oxygenation in patients suffering from hypoxia.

### SUMMARY

In some implementations, a system for intravascular oxygenation includes a catheter shaft, a vibratory member, an oxygen source and a check valve. The catheter shaft may have a wall that extends from a proximal end to a distal end along a longitudinal axis to form a lumen. The distal end may terminate in an atraumatic tip that seals off an interior space of the lumen from an adjacent exterior space. The wall may include a semi-porous membrane having a plurality of pores in the range of 5 nanometers and 10 micrometers. The vibratory member may be configured to produce and transmit to the wall mechanical vibration or high-frequency acoustic energy. The oxygen source may be configured to be coupled to the proximal end and deliver a flow of oxygen to an interior space for communication to the exterior space, through the plurality of pores. The check valve may be disposed between the oxygen source and the interior space and configured to stop the flow of oxygen to an interior space if a flow rate exceeds a first threshold or if a pressure falls below a second threshold.

In some implementations, the wall includes a plurality of folds that are parallel to the longitudinal axis and configured to increase a surface area of an exterior surface of the wall. An exterior surface of the wall may include a coating that is configured to repel a surface of a bubble formed at one of the plurality of pores. In some implementations, the coating is hydrophobic; in other implementations, the coating is hydrophilic.

In some implementations, the vibratory member is configured to produce mechanical vibration or high-frequency acoustic energy to release from the wall a bubble formed at one of the plurality of pores. In some implementations, the vibratory member includes a piezoelectric ring disposed at the anchor tab and around the catheter shaft. In some implementations, the vibratory member includes one or more reeds disposed in the interior space and configured to vibrate in response to the flow of oxygen.

In some implementations, the system further includes an anchor tab coupled to the proximal end and configured to secure the system to a patient when the catheter shaft is disposed in a vein of the patient.

In some implementations, the check valve includes a first safety feature that closes off communication between a downstream side and an upstream side when the flow rate exceeds the first threshold and a second safety feature that closes off communication between the downstream side and upstream side when the pressure falls below the second threshold. The first safety feature may include an orifice, a closure member that seals off the orifice upon contact with the same, and an elastic member configured to separate the closure member from the orifice whenever the flow rate exceeds the first threshold. The second safety feature may include an elastic flap valve configured to open only when the pressure is at or above the second threshold and remain closed when the pressure is below the second threshold.

In some implementations, a method of providing intravascular oxygenation to a patient includes providing (a) a catheter having (i) a shaft having a wall that extends from a proximal end to a distal end along a longitudinal axis to form a lumen, the distal end terminating in an atraumatic tip that seals off an interior space of the lumen from an adjacent exterior space; wherein the wall comprises a semi-porous membrane having a plurality of pores in the range of 5 nanometers and 10 micrometers; and (ii) a vibratory member configured to produce and transmit to the wall mechanical vibration or high-frequency acoustic energy; (b) an oxygen source configured to be coupled to the proximal end and deliver a flow of oxygen to the interior space for communication to the exterior space, through the plurality of pores; and (c) a check valve disposed between the oxygen source and the interior space and configured to stop the flow of oxygen to the interior space if a flow rate exceeds a first threshold or if a pressure falls below a second threshold; disposing the shaft in a vein of the patient; and coupling the oxygen source to the check valve, starting a flow of oxygen to the interior space, and activating the vibratory member to create oxygen microbubbles in the interior of the femoral vein of the patient. The vein may be at least one of a femoral vein, external jugular vein, internal jugular vein, subclavian vein, superior vena cava, or inferior vena cava.

In some implementations, a system for intravascular oxygenation includes a catheter shaft, a vibratory member, and an oxygen source. The catheter shaft may have a wall that extends from a proximal end to a distal end along a longitudinal axis to form a lumen. The distal end may terminate in an atraumatic tip that seals off an interior space of the lumen from an adjacent exterior space. The distal end may include a coiled spring whose coils are tightly disposed against adjacent coils. The vibratory member may be configured to produce and transmit via the wall, to the coiled spring, mechanical vibration or high-frequency acoustic energy. The oxygen source may be configured to be coupled to the proximal end and to deliver a flow of oxygen to an interior space for communication to the exterior space, through gaps that exist or are created between adjacent coils of the coiled spring.

In some implementations, the vibratory member is a piezoelectric ultrasonic transducer. The system may further include a horn disposed between the piezoelectric ultrasonic transducer and the catheter shaft.

The coils of the coiled spring may include a surface treatment of grooves, striations, a roughened surface, or a coating having different localized thicknesses. The system may further include a mass coupled to the distal end. The mass may be disposed in or adjacent to the atraumatic tip. The mass may include a rod that is affixed to the atraumatic tip or a portion of the distal end and configured to oscillate along a longitudinal axis of the distal end.

### BRIEF DESCRIPTION OF THE DRAWINGS

FIG. 1A illustrates an exemplary intravascular oxygenation system.

FIG. 1B illustrates a longitudinal cross-section of the catheter shaft shown in FIG. 1A.

FIG. 1C is a perspective view of one implementation of a catheter wall.

FIG. 2A is a perspective view of an exemplary semi-porous membrane.

FIGS. 2B-2D illustrate surfaces of exemplary semi-porous membranes.

FIG. 3A illustrates an exemplary vibratory member comprising a piezoelectric ring.

FIGS. 3B-3C illustrate exemplary vibratory members comprising one or more reeds.

FIG. 4 illustrates first and second safety features associated with an exemplary check valve.

FIG. 5 illustrates portions of the human circulatory system.

FIG. 6A illustrates another exemplary intravascular oxygenation system.

FIG. 6B depicts operation of the intravascular oxygenation system of FIG. 6A.

FIGS. 6C-6D illustrates additional details, in some implementations, of portions of the exemplary intravascular oxygenation system of FIG. 6A.

FIG. 7 is a flowchart of an exemplary method for intravascular oxygenation.

### DETAILED DESCRIPTION

Oxygen is an essential component for sustaining life. In healthy individuals, the body readily captures enough oxygen for healthy cell, tissue, and organ function; however, for those with certain respiratory conditions, such as hypoxemic respiratory failure, deprivation of this critical element can lead to severe respiratory distress, organ failure, and mortality without adequate intervention.

A variety of conditions can cause hypoxemia, including acute respiratory distress syndrome (ARDS), acute respiratory failure (ARF), physical trauma, chronic obstructive pulmonary disease (COPD), pulmonary fibrosis, sepsis, COVID-19, severe acute respiratory syndrome (SARS), lung cancer, congestive heart failure, and myocardial infarction, among others. ARDS and ARF are quite prevalent. ARF occurs when the respiratory system is unable to capture oxygen and remove carbon oxide from the bloodstream, while ARDS arises in those critically ill or who have significant lung injuries. Both impairments can result in hypoxia, which often proves fatal, even after administration of medical treatment.

Oxygen can be supplied to patients experiencing hypoxemia through mechanical ventilation (ML) or extacorporeal oxygentaiton (ECMO). However, both procedures are invasive, have side effects and high instances of mortality, and are exorbitantly expensive.

Described herein is an intravascular oxygenation system and method for delivering a less-invasive manner of oxygenation that may cost-effectively improve long-term safety for patients. In some implementations, the intravascular oxygenation system generates and delivers oxygen microbubbles directly to a patient's vasculature through a catheter system that is configured similarly to a peripherally inserted central catheter (PICC) line.

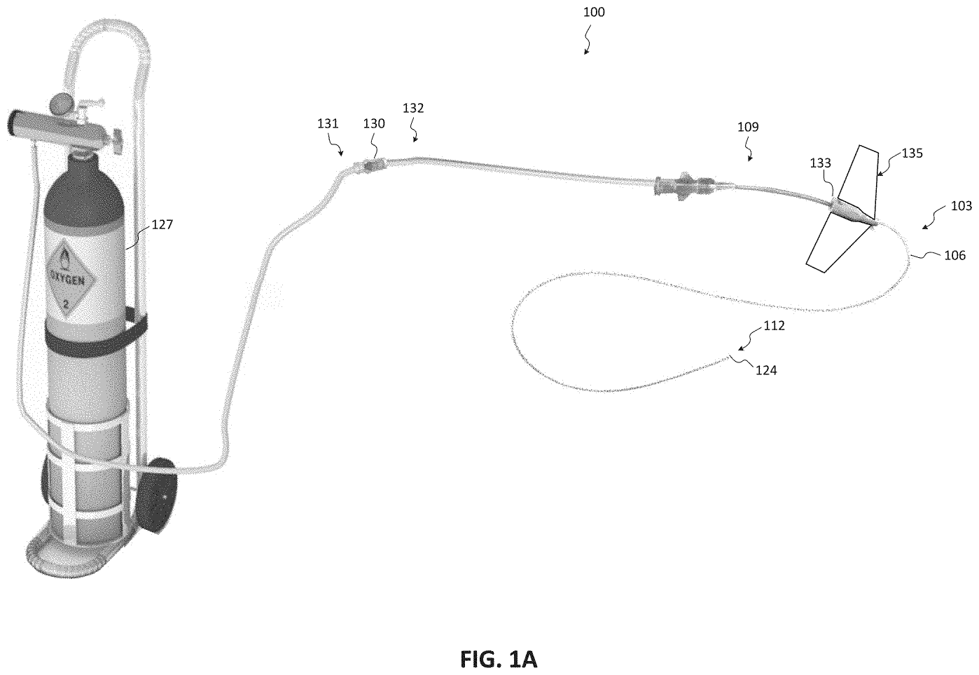

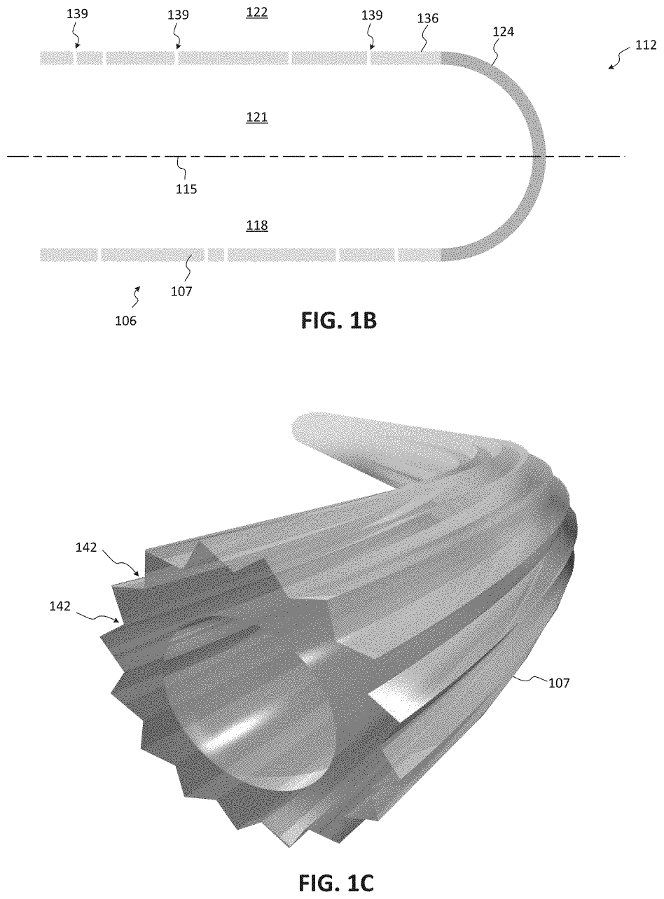

FIG. 1A illustrates a system 100 for intravascular oxygenation, in one implementation. As shown outside a patient (but in the configuration it would have when disposed inside the vasculature of a patient), the system 100 includes a catheter 103. The catheter 103 includes a shaft 106, which is configured to be disposed inside the patient's vasculature. The shaft 106 has a proximal end 109, which is configured to be outside the patient's body, close to an access site; and a distal end 112, which is configured to be disposed in the patient's vasculature.

To secure the catheter 103 to a patient in use, an anchor tab 135 may be provided. Such a tab 135 may be configured to be taped to the patient at an access site and clipped into the proximal end 109 of the catheter 103 in order to secure it.

In some implementations, with reference to FIG. 1B, the shaft 106 comprises a wall 107 that extends from the proximal end 109 to a distal end 112 along a longitudinal axis 115 to form an internal lumen 118. In contrast to a PICC line, the shaft 106 terminates in an atraumatic tip 124 that seals off an interior space 121 of the lumen 118. In some implementations, the atraumatic tip 124 comprises a smooth, low-friction material that is configured to slide easily along an interior of a vessel without catching or puncturing the vessel.

In some implementations, the wall 107 is a semi-porous membrane 136 having a plurality of microscopic pores 139 that are configured to release pressurized oxygen from the interior space 121 into an adjacent exterior space 122, through the formation of microbubbles of oxygen.

The pores 139 may be configured to release oxygen from the interior space 121 in a manner that creates microbubbles that facilitate an efficient and timely transfer of oxygen to deoxygenated blood, while at the same time maintaining safe bubble size to minimize the creation of air emboli. For many patients, bubbles larger than 10 micrometers may be filtered out (ruptured and absorbed, in many cases) by the pulmonary structure of the lungs. However, bubbles that are significantly larger than 10 micrometers may be associated with a higher risk of aggregation or coalescence in a manner that could cause an air embolism. Accordingly, in some implementations, the pores 139 are configured to create microbubbles in the range of 5 nanometers to 10 micrometers.

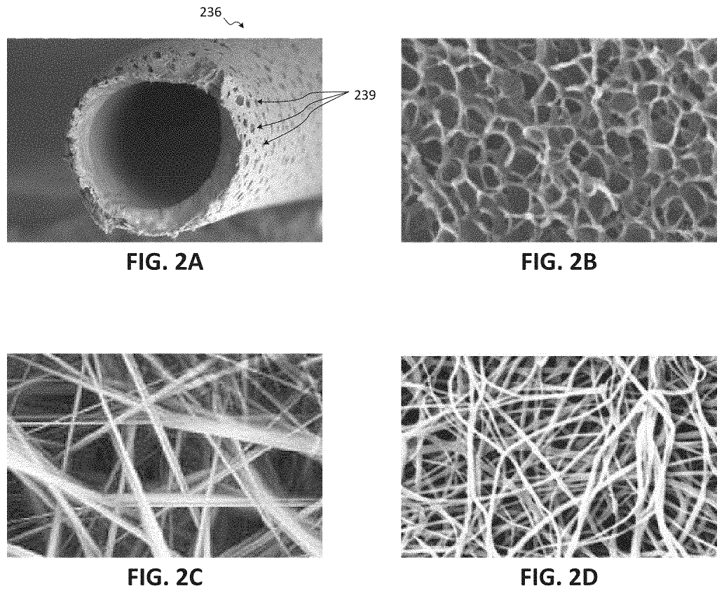

In some implementations, as shown in FIG. 2A, pores 239 are formed in a semi-porous membrane 236 that is configured similar to membranes employed for dialysis (e.g., hemodialysis membranes). In some implementations, the semi-porous membrane 236 comprises polysufone (PSf), polyethersulfone (PES), polyamide (PA), or cellulose acetate (CA). In some implementations, one surface of the membrane 236 comprises a dense polymer layer with nanometer-sized pores adjacent a more porous sublayer having voids separated by polymer fibers.

In some implementations, as shown in FIG. 2B, a portion of the membrane may comprise an open-cell biocompatible foam structure. As shown, the cell structure is relatively large. In other implementations, individual cells may be smaller, and the cells may be disposed within a more solid, less porous substrate. In general, size of the cells in a foam structure can influence the size and number of microbubbles that form on an exterior of the semi-porous membrane.

In some implementations, as shown in FIG. 2C, a portion of the membrane may comprise a plurality of fibers in a relatively random crossing configuration, such as is common in electro-spun mats of nanofibers used in high-efficiency filtration applications. By varying the thickness of the overall mat, the size of the fibers, and the density of the fibers, it may be possible to control size and numbers of microbubbles that form on the surface of such a membrane. For example, FIG. 2D illustrates a denser arrangement of smaller fibers.

In general, various arrangements and types of fibers, foams and membranes are possible, using known techniques for their formation, and using established biocompatible materials. In some implementations, regardless of the precise construction, membranes may be formed in manners similar to high-efficiency particulate filters, hemodialysis filters or a combination thereof; and the manufacturing process may be controlled such that circuitous conduits are formed through the thickness of the membrane, such that pressured oxygen on one side of the membrane can be forced through the circuitous conduits to form microbubbles on the opposite side of the membrane.

In some implementations, additional features may be provided in a system to facilitate creation of optimally sized microbubbles and prevent coalescence or aggregation of those bubbles. For example, in some implementations, a porous membrane may be treated with a coating that is designed to facilitate release of microbubbles from an exterior surface of the membrane shortly after the microbubbles are formed. More particularly, individual fibers, such as those shown in FIG. 2C and FIG. 2D, or an overall external surface of a foam material, such as the one shown in FIG. 2A, may be treated with a coating that either repels or attracts water, plasma or other blood constituents; or reduces surface friction or enhances lubricity. In this manner, as microbubbles are formed during operation of the system, the microbubbles may be repelled by the surface of the membrane and carried away by the intravascular flow of blood adjacent the membrane.

A separate mechanism for vibrating the porous membrane 136 may be provided to dislodge microbubbles shortly after they are formed. In some implementations, a vibratory member 133 (see FIG. 1A), such as a piezoelectric device, may be configured to produce and transmit to the catheter 103 mechanical vibration or high-frequency acoustic energy. In some implementations, the mechanical vibration or acoustic energy may agitate the blood boundary layer around each microbubble to increase oxygen absorption. In some implementations, bubbles forming on the surface of the semi-porous membrane 136 may be dislodged by the vibration or acoustic energy and carried away into the bloodstream.

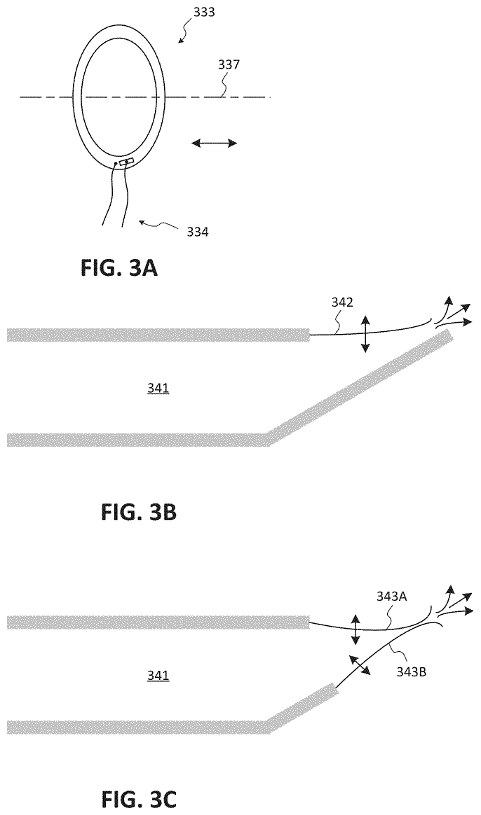

FIG. 3A illustrates an exemplary piezoelectric ring 333 that may be employed to produce high-frequency acoustic energy that can be transmitted or conducted to a semi-porous membrane to dislodge microbubbles. The piezoelectric ring 333 can be configured to, when energized (e.g., by supplying a voltage to leads 334), vibrate perpendicular to an axis 337. In this manner, the vibrations (which may comprise high-frequency acoustic energy) may be transmitted in a longitudinal direction of the shaft 106 of the catheter 103 shown in FIG. 1A. In some implementations, the piezoelectric ring 333 comprises a thin ceramic or composite device polarized axially and radially to produce high-frequency vibrations.

A frequency and magnitude of vibration may be employed to minimize any sensation by the patient, while still actuating the wall 107 sufficiently to dislodge microbubbles as they form. Such a piezoelectric ring 333 may be disposed on or near the anchor tab 135 shown in FIG. 1A (see element 133), to facilitate easy connection to a voltage supply external to the patient.

In some implementations, another method for generating mechanical vibration or high-frequency acoustic energy may be employed. For example, as depicted in FIG. 3B., oxygen flowing into the catheter 103 may be routed through a narrow channel 341 having an opening closed with a flexible reed 342. As the oxygen exits the narrow channel 341, the reed 342 may be configured to vibrate in a manner that generates oscillating acoustic energy that can be transmitted to a wall 107 of the shaft 106. The material and elasticity of the reed 342, and dimensions of the channel 341 relative to the flow of oxygen can be selected to achieve the desired level of acoustic energy. In some implementations, as depicted in FIG. 3C, the narrow channel 341 may be closed with two flexible reeds 343A and 343B, such that that reeds 343A and 343B close against each other and open away from each other.

Returning to FIG. 1A, the system 100 further includes an oxygen source 127 that is configured to be coupled to the proximal end 109 and deliver a flow of oxygen to an interior space 121 for communication to the exterior space 122 (see FIG. 1D), through the plurality of pores 139. In some implementations, the oxygen source 127 is a canister or tank, as shown. In other implementations, the oxygen source may include a plumbed, building-wide oxygen distribution system, as is common in most hospitals and medical facilitates.

Regardless of its precise design, the oxygen source 127 may be coupled to the catheter 103 through a check valve 130. The check valve may be configured to maintain a positive pressure within the interior space 121 (e.g., to prevent backflow of any pressurized gas if the pressure on an upstream side 131 of the check valve 130 falls below a pressure of the downstream side 132 of the check valve 130. In addition, the check valve 130 may be configured to stop the flow of oxygen if a flow rate or pressure exceeds a safe threshold, to minimize any risk of rupture of the catheter 103 while it is inside a patient. Other flow-control, pressure-control, or filtering devices (not shown) may also be disposed between the oxygen source 127 and the catheter 103. For example, mechanical or chemical filters may be provided to prevent any particulate matter that may be in the stream of oxygen from the oxygen source 127 from entering the catheter, or to remove any gaseous other impurities that may be present in that stream of oxygen.

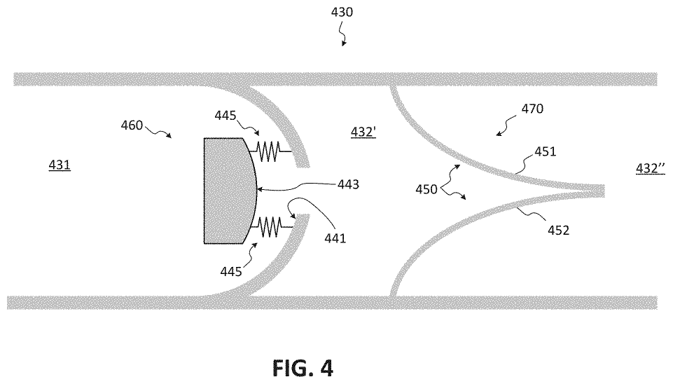

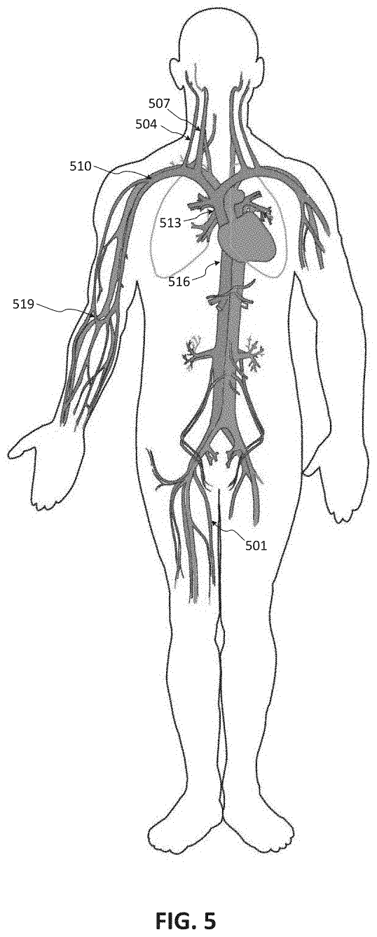

FIG. 4 illustrates components of an exemplary check valve 430. As shown, the check valve 430 comprises a first safety feature 460 that closes off communication between an upstream side 431 and a downstream side 432′ whenever the upstream pressure or flow exceeds a safe threshold value. As depicted functionally, the first safety feature 460 can include a contoured opening 441 and a correspondingly contoured valve member 443. Under normal operation, a separation may be maintained between the contoured opening 441 and the valve member 443 by elastic members 445 (e.g., springs, in some implementations). However, when the pressure or flow on an upstream side 431 exceeds a safe threshold, that pressure or flow impinges on the valve member 443 with sufficient force to overcome the spring force of the elastic members 445, thereby pushing the valve member 443 against the contoured opening 441 and closing off flow. This description and corresponding figure are merely exemplary; various designs for check valves are known, and many may be suitable for this application.

In some implementations, the check valve 430 comprises a second safety feature 470 that closes off communication between an intermediate upstream side 432′ and a downstream side 432″. As shown in cross-section, the second safety feature 470 may comprise an elastic membrane or septum 450 having a first flap 451 and a second flap 452. Under no-flow or low-pressure scenarios, elastic force of the membrane 450 may keep the first flap 451 in contact with the second flap 452, essentially sealing off the upstream 432′ and downstream 432″ sides of the safety feature 470. At higher flows or pressures on the upstream side 432′, the force of such flow/pressure may cause separation between the first flap 451 and the second flap 452, facilitating communication through the second safety feature 470.

As depicted, the geometry of the second safety feature 470 may be such that backflow or back pressure from the downstream side 432″ does not create a separate between the first flap 451 and second flap 452; thus a backflow or back pressure may be prevented by the second safety feature 470. As with the first safety feature 460, this description and corresponding functional illustration are merely exemplary. Many check valve designs for ensuring minimal flow and pressure and for preventing backflow or back pressure are known and could be adopted here.

Provided that pressure and flow are safely controlled, as just described, and provided that microbubbles do not coalesce or aggregate, it is advantageous in many implementations to maximize the quantity of microbubbles generated, to thereby increase the level of intravascular oxygenation. Thus, it may be advantageous to maximize the surface area of the wall 107 of the catheter 103. FIG. 1C is a perspective view of one implementation in which surface area of the wall 107 can be maximized. Specifically, as shown, folds 142 are formed in the wall 107. In some implementations, such folds 142 are parallel to a longitudinal axis of the shaft 106.

Turning to FIG. 5, exemplary access points through which microbubbles can be delivered to a patient for intravascular oxygenation are now described. In some implementations, one way to introduce such microbubbles is to deliver them ultimately into the superior vena cava 513 or inferior vena cava 516. There are several common access points through which oxygen microbubbles can be so introduced. Common among them is the median cubital vein 519 of the right arm. From here, blood flows through the basilic vein, axillary vein, subclavian vein, and into the superior vena cava 513. Alternative paths to the superior vena cava 513 are the external jugular vein 504 or internal jugular vein 507—both of which drain into the brachiocephalic vein prior to reaching the superior vena cava 513—or the subclavian vein 510. An alternative inferior route includes the femoral vein 501, which flows into the inferior vena cava 516. Other routes to the superior vena cava 513 and inferior vena cava 516 are possible.

In some implementations, access through the femoral vein 501 may be preferable, given its diameter (facilitating a larger bore catheter than may be possible in other veins), length between standard access point on the leg and inferior vena cava (facilitating microbubble generation over a relatively long distance and corresponding surface area), and relatively straight path (minimizing potential trauma to the vasculature that may be brought about by navigating the catheter through various turns and vessel junctions).

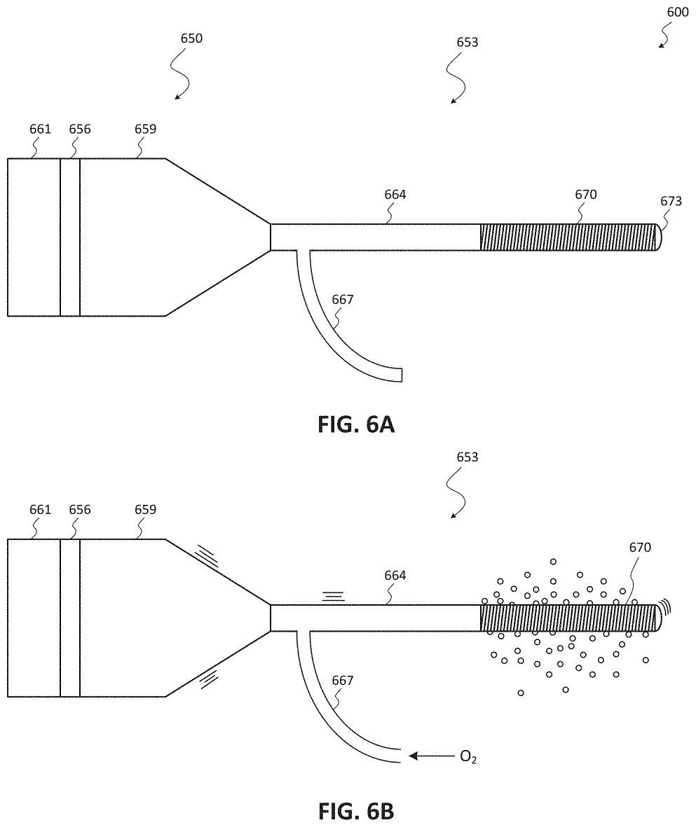

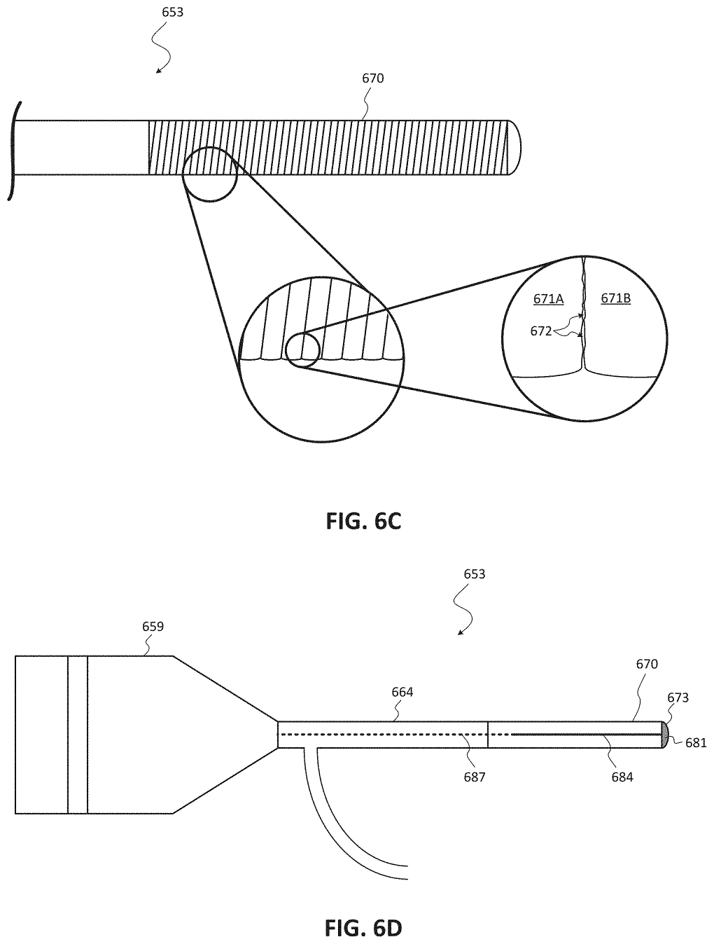

FIG. 6A illustrates a system 600 for intravascular oxygenation, in another implementation. As shown, the system 600 includes a proximal portion 650, which remains outside of a patient; and a distal portion 653, which is configured to be disposed in the vasculature of a patient. To assist in delivering the distal portion 653 to the vasculature of a patient, the system can include an atraumatic tip 673 configured to slide smoothly and with minimal friction within the vasculature of the patient.

In the implementation shown, the distal portion 653 includes a lumen 664 and input port 667 into which a gas, such as oxygen, may be delivered to an interior of the lumen 664 and ultimately to a distal end 670. In some implementations, the lumen 664 is a hypotube or other lumen structure with relatively rigid walls that are capable of transmitting acoustic energy longitudinally but with sufficient flexibility to facilitate navigation of curved human vasculature.

In some implementations, the lumen 664 is configured to transmit acoustic energy (e.g., in the form of mechanical vibrations) without significant loss, so as to facilitate formation of standing waves within the lumen 664 and/or distal end 670—e.g., when energy is generated at the ultrasonic transducer 656, directed in one direction by the horn 659, transmitted by the lumen 664, and reflected back by the distal end 670 or the atraumatic tip 673 of the distal end 670.

In some implementations, the distal end 670 is configured to facilitate release of the gas introduced at the input port 667 into a region exterior to the lumen 664 and distal portion 670. For example, in an implementation in which the distal portion 653 is disposed in the vasculature of a patient, and oxygen is introduced into the input port 667, the oxygen may be released (e.g., in the form of microbubbles) from the distal end 670, as depicted in FIG. 6B.

In some implementations, the distal end 670 comprises a coiled spring whose coils are tightly disposed against adjacent coils. Miniscule gaps may exist (or be temporarily created under pressure or by vibrations) between adjacent coils in a manner that enables pressurized gas to escape through the miniscule gaps. For example, with reference to FIG. 6C—which shows a magnified view of one coil 671A and an adjacent coil 671B—a plurality of gaps 672 may exist. In some implementations, such gaps 672 exist naturally (e.g., as a function of variations in manufacture of the coils 671A and 671B—such as minor variations in thickness, surface smoothness or elasticity); in other implementations, such gaps 672 may be designed into the coils 671A and 671B—for example, through application of one or more surface treatments, such as scribing or other application of grooves or striations, roughening of the surface to add irregularities or variations in surface contour, application of coatings having different localized thicknesses, etc.

In implementations in which the distal portion 653 is disposed in a liquid medium (e.g., the vasculature of a human patient, through which blood may be continuously flowing), the pressurized gas (e.g., oxygen) can escape in the form of microbubbles. To facilitate release of any such microbubbles while such microbubbles are relatively small, a vibratory member, such as an ultrasonic transducer 656, may be provided in the proximal portion 650. In some implementations, the ultrasonic transducer 656 is a piezoelectric device that generates ultrasonic energy in the form of high-frequency mechanical vibrations.

A horn 659 may be provided adjacent the transducer 656 to perform one or more functions: transferring ultrasonic energy from the transducer 656 to the lumen 664, increasing the amplitude of the ultrasonic energy provided by the transducer 656 (e.g., increasing the oscillation displacement amplitude), and tuning frequency. In general, an exemplary horn, like the horn 659, has a decreasing cross-sectional area along its longitude, which causes waves propagating through the horn 659 to increase in amplitude as they move from greater cross-sectional area to lesser cross-sectional area (e.g., left to right, in FIGS. 6A and 6B). The physical dimensions of the horn 659 also influence frequency of waves propagating through the horn 659. That is, the taper of the horn 659 may be machined, as well as the length of the horn 659—for example, to tune the frequency output of the horn 659.

The horn 659 may take various forms—including having a stepped, exponential, conical, catenoidal, or other longitudinal cross-sectional shape; a round, rectangular, or other transverse cross-sectional shape; one or more distinct elements with different longitudinal cross-sectional profiles, with various possible types of transitional elements between multiple distinct elements; and comprising various materials, such as a titanium alloy (e.g., Ti6Al4V), a stainless steel (e.g., 440C), an aluminum alloy, a powdered metal, or another suitable material.

A back mass 661 may also be provided as a stable “base” for the ultrasonic transducer 656. That is, the back mass 661 (e.g., via inertia) may cause ultrasonic energy from the transducer 656 to be primarily directed into the horn 659, rather than allowing the ultrasonic transducer 656 to simply vibrate.

In operation, as depicted in FIG. 6B, pressurized gas, such as oxygen, can be provided at the input port 667. From the input port 667, the gas can flow through the lumen 664, to the distal end 670, where the gas can escape in the form of microbubbles. When the transducer 656 is activated while gas is flowing from the input port 667 to the distal end 670, high-frequency mechanical energy may be provided by the transducer 656, focused and transferred by the horn 659 to the lumen 664, and transmitted by the lumen 664 to the distal end 670, where the mechanical vibration may then dislodge microbubbles as they are formed on the surface of the distal end 670, prior to the bubbles becoming larger than desired. Thus, in some implementations, by varying pressure of gas delivered at the input port 667, parameters of the ultrasonic energy (e.g., frequency and amplitude of mechanical vibrations), and parameters of the distal end 670 (e.g., material type, dimensions, elastic force, surface treatment, etc. of the coiled spring), it may be possible to customize the size and quantity of microbubbles formed and released.

In some implementations, as depicted in the cross section shown in FIG. 6D, additional mass may be added to the distal portion 653 to amplify the effect of the mechanical vibrations at the distal end 670. For example, a mass 681 may be added to, or adjacent to, the atraumatic tip 673. As another example, a mass such as a rod 684 may be affixed to the atraumatic tip 673 or a portion of the distal end 670, such that the rod can oscillate along a longitudinal axis of the distal end 670. In such implementations, the additional mass 684 and/or 681—in conjunction, in some implementations, with a standing wave, as described above—may more effectively dislodge bubbles from a surface of the distal end 670, or otherwise facilitate tuning of when such bubbles are dislodged.

In some implementations, elastic elements, such as an elastic element 687, may also be employed to facilitate further tuning (e,g., by providing some dampening of vibrations) of the kinetic energy of the distal end 670. In some implementations, as shown, the elastic element 687 may be anchored to the horn 659; in other implementations, the elastic element 687 may be anchored to a portion of the lumen 664 or the distal end 670.

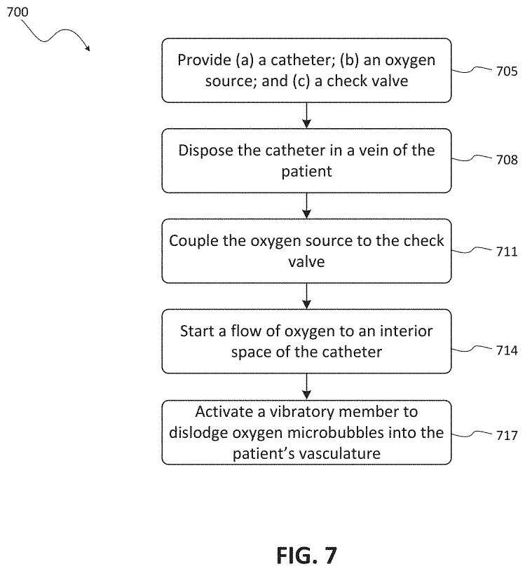

FIG. 7 depicts an exemplary method 700 for providing intravascular oxygenation to a patient. The method 700 comprises providing (705) a catheter, oxygen source and a check valve. For example, the system 100 shown in FIG. 1A could be provided.

The method 700 further comprises disposing (708) the catheter in a vein of a patient. For example, the shaft 106 of the system 100 could be disposed in a patient in need of intravascular oxygenation—specifically, for example, in the femoral vein of such a patient. In some implementations, the shaft 106 may be inserted through a process similar to that used to install a PICC line—namely, by (a) injecting a large bore needle containing a guidewire into the patient's vein; (b) removing the needle and inserting an introducer over the guidewire; (c) removing the guidewire and inserting the shaft 106 through the introducer into the patient's vein; (d) peeling away the removable introducer; and (e) fastening the shaft in place externally using an anchor tab (e.g., anchor tab 135).

As another example, the distal portion 653 of a system 600 may be disposed in a patient in need of intravascular oxygenation. For example, in an ambulatory setting, the distal portion 653—in particular, the distal end 670—may be disposed in the vasculature of the patient, e.g., via an introducer or small incision, leveraging the atraumatic tip 673 to guide insertion. In some implementations, the distal portion 653 may be disposed in the interior or exterior jugular vein of a patient; in other implementations, the distal portion 653 may be disposed in a vein of the patient's arm or leg (e.g., median cubital vein, basilic vein, axillary vein, subclavian vein, femoral vein, etc.).

The method 700 further comprises coupling (711) an oxygen source to the check valve. For example, with reference to FIG. 1A, the oxygen source 127 may be coupled to the check valve 130, which in turn, may be coupled to the catheter 103.

The method 700 further comprises starting (714) a flow of oxygen. For example, the oxygen source 127 shown in FIG. 1A may be opened at a safe pressure and flow, such that oxygen flows through the check valve and into the catheter 103. More specifically, the oxygen can flow into an interior space 121 (see FIG. 1B) of a lumen 118 formed by the shaft 106 of the catheter; and the oxygen may then flow out through micropores 139, to create microbubbles in a space exterior (e.g., space 122)—for example, in the vasculature of a patient.

In some implementations, the method 700 further comprises activating (717) a vibratory member to assist in dislodging microbubbles from an exterior wall of the catheter, to minimize the coalescence or aggregation of such microbubbles and to promote oxygenation of the adjacent blood intravascularly. For example, in some implementations, a piezoelectric ring 333 (see FIG. 3A) may be actuated to create high-frequency acoustic energy. As another example, a reed 342 or reeds 343A and 343B may be employed (see FIGS. 3B, 3C) to generate similar high-frequency acoustic energy. As another example, an ultrasonic transducer 656 (see FIGS. 6A and 7B) may be activated.

Many other variations are possible, and modifications may be made to adapt a particular situation or material to the teachings provided herein without departing from the essential scope thereof. Therefore, it is intended that the scope include all aspects falling within the scope of the appended claims.

What is claimed is:

1. A system for intravascular oxygenation, the system comprising:
a catheter shaft having a wall that extends from a proximal end to a distal end along a longitudinal axis to form a lumen, the distal end terminating in an atraumatic tip that seals off an interior space of the lumen from an adjacent exterior space; wherein the wall comprises a semi-porous membrane having a plurality of pores in the range of 5 nanometers and 10 micrometers;
a vibratory member configured to produce and transmit to the wall mechanical vibration or high-frequency acoustic energy;
an oxygen source configured to be coupled to the proximal end and deliver a flow of oxygen to an interior space for communication to the exterior space, through the plurality of pores; and
a check valve disposed between the oxygen source and the interior space and configured to stop the flow of oxygen to an interior space if a flow rate exceeds a first threshold or if a pressure falls below a second threshold.

2. The system of claim 1, wherein the wall comprises a plurality of folds that are parallel to the longitudinal axis and configured to increase a surface area of an exterior surface of the wall.

3. The system of claim 1, wherein an exterior surface of the wall comprises a coating that is configured to repel a surface of a bubble formed at one of the plurality of pores.

4. The system of claim 3, wherein the coating is a hydrophobic coating.

5. The system of claim 3, wherein the coating is a hydrophilic coating.

6. The system of claim 1, wherein the vibratory member is configured to produce mechanical vibration or high-frequency acoustic energy to release from the wall a bubble formed at one of the plurality of pores.

7. The system of claim 1, further comprising an anchor tab coupled to the proximal end and configured to secure the system to a patient when the catheter shaft is disposed in a vein of the patient.

8. The system of claim 7, wherein the vibratory member comprises a piezoelectric ring disposed at the anchor tab and around the catheter shaft.

9. The system of claim 1, wherein the vibratory member comprises one or more reeds disposed in the interior space and configured to vibrate in response to the flow of oxygen.

10. The system of claim 1, wherein the check valve comprises a first safety feature that closes off communication between a downstream side and an upstream side when the flow rate exceeds the first threshold and a second safety feature that closes off communication between the downstream side and upstream side when the pressure falls below the second threshold.

11. The system of claim 10, wherein the first safety feature comprises an orifice, a closure member that seals off the orifice upon contact with the same, and an elastic member configured to separate the closure member from the orifice whenever the flow rate exceeds the first threshold.

12. The system of claim 10, wherein the second safety feature comprises an elastic flap valve configured to open only when the pressure is at or above the second threshold and remain closed when the pressure is below the second threshold.

13. A method of providing intravascular oxygenation to a patient, the method comprising:
providing (a) a catheter having (i) a shaft having a wall that extends from a proximal end to a distal end along a longitudinal axis to form a lumen, the distal end terminating in an atraumatic tip that seals off an interior space of the lumen from an adjacent exterior space; wherein the wall comprises a semi-porous membrane having a plurality of pores in the range of 5 nanometers and 10 micrometers; and (ii) a vibratory member configured to produce and transmit to the wall mechanical vibration or high-frequency acoustic energy; (b) an oxygen source configured to be coupled to the proximal end and deliver a flow of oxygen to the interior space for communication to the exterior space, through the plurality of pores; and (c) a check valve disposed between the oxygen source and the interior space and configured to stop the flow of oxygen to the interior space if a flow rate exceeds a first threshold or if a pressure falls below a second threshold;
disposing the shaft in a vein of the patient; and
coupling the oxygen source to the check valve, starting a flow of oxygen to the interior space, and activating the vibratory member to create oxygen microbubbles in the interior of the femoral vein of the patient.

14. The method of claim 13, wherein the vein is at least one of a femoral vein, external jugular vein, internal jugular vein, subclavian vein, superior vena cava, or inferior vena cava.

15. A system for intravascular oxygenation, the system comprising:
a catheter shaft having a wall that extends from a proximal end to a distal end along a longitudinal axis to form a lumen, the distal end terminating in an atraumatic tip that seals off an interior space of the lumen from an adjacent exterior space; wherein the distal end comprises a coiled spring whose coils are tightly disposed against adjacent coils;
a vibratory member configured to produce and transmit via the wall, to the coiled spring, mechanical vibration or high-frequency acoustic energy; and
an oxygen source configured to be coupled to the proximal end and to deliver a flow of oxygen to an interior space for communication to the exterior space, through gaps that exist or are created between adjacent coils of the coiled spring.

16. The system of claim 15, wherein the vibratory member comprises a piezoelectric ultrasonic transducer.

17. The system of claim 16, further comprising a horn disposed between the piezoelectric ultrasonic transducer and the catheter shaft.

18. The system of claim 16, wherein the coils of the coiled spring comprise a surface treatment comprising grooves, striations, a roughened surface, or a coating having different localized thicknesses.

19. The system of claim 16, further comprising a mass coupled to the distal end.

20. The system of claim 19, wherein the mass is disposed in or adjacent to the atraumatic tip, or where the mass comprises a rod that is affixed to the atraumatic tip or a portion of the distal end and configured to oscillate along a longitudinal axis of the distal end.
